(19) United States
(12) Patent Application Publication
(10) Pub. No.: US 2026/0000474 A1
(43) Pub. Date: Jan. 1, 2026

(54) ROBOTIC SURGERY SYSTEM LAYOUTS AND RELATED METHODS

(71) Applicant: Monogram Technologies Inc., Austin, TX (US)

(72) Inventors: Pedro Alfonso PATLAN ROSALES, Buda, TX (US); Sanath VURELLI, Waltham, MA (US); Aeshan ALI, Austin, TX (US); Ani NAYAK, Dripping Springs, TX (US); Kamran SHAMAEI, Austin, TX (US)

(73) Assignee: Monogram Technologies Inc., Austin, TX (US)

(21) Appl. No.: 19/320,637

(22) Filed: Sep. 5, 2025

Related U.S. Application Data
(63) Continuation of application No. PCT/US2024/018882, filed on Mar. 7, 2024.
(60) Provisional application No. 63/488,973, filed on Mar. 7, 2023.

(51) Int. Cl. A61B 34/32; A61B 17/00; A61B 17/15; A61B 34/10; A61B 34/20; A61B 34/37; A61B 50/13; A61F 2/46

(52) U.S. Cl. CPC A61B 34/32; A61B 17/155; A61B 17/157; A61B 34/37; A61B 50/13; A61F 2/461; A61B 2017/00973; A61B 2034/107; A61B 2034/2057; A61B 2034/2072; A61F 2002/4632

(57) ABSTRACT

Autonomous surgical robotic systems and methods with advantages component layout. The system includes a patient support, an active cutting system positioned adjacent to the patient support on a first lateral side of the patient support, an electronic controller positioned adjacent to the patient support on a second lateral side of the patient support that opposes the first lateral side of the patient support. The system further includes at least one fixed patient tracking array configured to couple to a patient on the patient support at a fixed positional relationship to a surgical site of the patient, and a navigation system with at least one positional tracking camera system positioned on the first lateral side of the patient support.

### CROSS-REFERENCE TO RELATED APPLICATIONS

This application is a bypass continuation of PCT Application No. PCT/US2024/018882, filed on Mar. 7, 2024, and entitled “Robotic Surgery System Layouts and Related Methods,” which claims the benefit of priority of U.S. Provisional Application No. 63/488,973, entitled Robotic Surgery System Layouts and Related Methods, filed on Mar. 7, 2023, the entirety of which is expressly incorporated herein by reference.

### TECHNICAL FIELD

The following disclosure relates generally to the layout or morphology of components of robotic surgery systems. More particularly, the following disclosure relates to the layouts of components of autonomous surgical robotic systems in the operating theatre, and related autonomous robotic surgical methods.

### BACKGROUND OF THE INVENTION

Recently, surgical robots have become available which can partially control powered cutting tools for orthopedic surgical procedures. Such surgical robots typically include an articulated robotic arm that facilitates the gross movement of a cutting tool, such as along cutting pathways.

The current state-of-the-art in orthopedic surgical robots are haptic robots. Haptic orthopedic surgical robots rely on a user to provide the gross movement of the cutting tool (and typically a drive mechanism that operates the cutting tool along a cutting pathway along which the cutting tool is designed to cut) along a cut path. These orthopedic surgical robots are designed for user initiated cutting such that the robot itself is not actively or autonomously executing the cuts, but rather the user guides the cutting tool within predefined spatial zones that the robot “allows” the cutting to move within.

Such haptic current surgical robots are thus configured as hand-guided instruments that power the cutting tool and, at best, assist a user in translating the cutting tool to (and through) a patient, but require a user to manually move and direct the cutting tool along its cutting pathway (i.e., the robot is not actively or autonomously executing the cuts). For example, some such haptic surgical robots include a handle and a trigger that a user manually utilizes to move and direct an active cutting tool along its cutting pathway. The user (e.g., a surgeon) must thereby manually provide inputs to the robot by physically moving it.

It is noted that some such haptic robotic cutting systems may electronically or autonomously define a cutting area/zone and a non-cutting area/zone, and actively prevent a user form translating the cutting tool into/through the non-cutting area to protect areas of a patient that should not be cut (e.g., accidently). However, such robotic systems do not actively or autonomously translate the cutting tool through the cutting area, as the user must physically move the cutting tool along the cut paths in the cutting area.

Haptic robotic cutting systems thereby require one or both of a user's hands to manually physically guide the cutting tool (and drive mechanism) to and through cut paths. In surgical applications, it is often necessary to surgically cut or resect a bone, cartilage and/or other tissue of a patient (e.g., a mammalian patient), such as during a surgical procedure. As haptic robotic cutting systems require user initiated cutting, the systems are relatively demanding on users (e.g., surgeons), limits a user's ability to perform other tasks, and the robotic system often physically interferes with access to the patient. For example, as the cutting tool must be physically guided by the user, the articulated robotic arm, typically mounted on a cart or like movable base, is positioned on the same side of the patient table or other supporting construct (and thus a patient thereon) as the user. Because the articulated robotic arm must be so close to the patient and the user so that the user can manually guide the cutting tool, such robots have a tendency to collide with the patient, user and/or assistant user in the operating theater. Also, the cart or base of the articulated robotic arm, and/or the articulated robotic arm itself, crowds the user and interferes with his/her movement and visual clarity.

Further, prior robotic surgical systems position a navigation system, typically mounted on a cart or like movable base, on an opposite side of the patient/patient table as the user and the articulated robotic arm. The navigation system include one or more cameras, screens/graphical displays or interfaces, and potentially computer/processing equipment, and track the positions of the cutting tool and the patent to provide the computer-aided cutting zones and boundaries, as described above. In such arrangement, personnel in the operating theater often occlude the tracking camera(s) as that side of the patent table/patient is open, and the user needs to turn his/her head or turn their body to view the screen(s)/display(s) of the navigation system. The positioning of the navigation system also spaces the tracking camera(s) a significant distance from the surgical site, potentially introducing errors in tracking.

Fully autonomous or active robotic systems and related methods that autonomously follow determined cut paths, without a user physically engaging and guiding the cutting tool, or drive mechanism, are desirable. For example, autonomous or active orthopedic and/or surgical robotic systems and related methods that autonomously follow determined cut paths would be advantageous to allow the user (e.g., surgeon) to perform other tasks.

The present disclosure provides improved robotic systems and related methods that autonomously/actively follow determined cut paths without a user physically engaging and guiding the cutting tool (or drive mechanism), and include an intuitive user controller for a limited amount of control of the cutting parameters or process by the user. The improved autonomous/active robotic systems provide for optimized efficient (potentially pre-planned) cut paths, allow the user to use both of their hands for other tasks, and free up working space about the robotic system, while providing a user with some level of control of the cutting parameters or process to ensure safe, proper, and efficient cuts. The improved robotic systems and related methods also include component layouts in the surgical theater that unexpectedly improve the usability and performance of the systems.

While certain aspects of conventional technologies have been discussed to facilitate disclosure of Applicant's inventions, the Applicant in no way disclaims these technical aspects, and it is contemplated that the inventions may encompass one or more conventional technical aspects.

In this disclosure, where a document, act or item of knowledge is referred to or discussed, this reference or discussion is not an admission that the document, act or item of knowledge or any combination thereof was, at the priority date, publicly available, known to the public, part of common general knowledge, or otherwise constitutes prior art under the applicable statutory provisions; or is known to be relevant to an attempt to solve any problem with which this specification is concerned.

### SUMMARY OF THE INVENTION

The present inventions may address one or more of the problems and deficiencies of current surgical robots, surgical robot system components and related surgical methods. However, it is contemplated that the inventions may prove useful in addressing other problems and deficiencies in a number of technical areas. Therefore, the claimed invention(s) should not necessarily be construed as limited to addressing any of the particular problems or deficiencies discussed herein.

The present disclosure is generally directed to surgical robots, surgical robotic systems and related surgical methods, and in particular orthopedic surgical robots, orthopedic surgical robotic systems and related orthopedic surgical methods, that are arranged in the surgical theater in a layout that unexpectedly provides advantageous usability and enhanced performance of the system. The surgical robotic systems include a controller that allows a user to electronically control parameters of movement of a cutting tool along a prescribed or determined cut path. The controller provides intuitive user control of the active robot along a prescribed or determined cut path thereof.

Further, such surgical robotic systems include a robotic cutting system and a navigation system, that monitors and controls the movement of a cutting tool of the robotic cutting system, situated relative to a patient that unexpectedly advantageously minimizes occlusion by users of one or more tracking camera of the navigation system. The layout of the robotic cutting system and a navigation system relative to the patient also unexpectedly advantageously improves tracking and cutting tool positioning accuracy. The layout of the robotic cutting system and a navigation system relative to the patient also unexpectedly advantageously minimizes the likelihood of the robotic arm from colliding with a user or the patient. The layout of the robotic cutting system and a navigation system relative to the patient also unexpectedly advantageously improves the accessibility and breadth of the user's workspace area. The layout of the robotic cutting system and a navigation system relative to the patient also unexpectedly advantageously reduces the likelihood and/or volume of splatter from a cutting operation onto tracking areas that are utilized by the navigational system to determine the position/orientation of the cutting tool and/or a surgical site of the patient. The layout of the robotic cutting system and a navigation system relative to the patient also unexpectedly advantageously allows a user of the robotic cutting system to easily access and reposition one or more display and/or camera of the navigation system. The layout of the robotic cutting system and a navigation system relative to the patient also unexpectedly advantageously provides an unobstructed view path of one or more displays of the navigation system in line with a user's line of sight when the user faces the surgical site.

In one aspect, the present disclosure provides surgical robotic system, comprising: a patient support configured to physically support a surgical site of a patient at a particular location; an active cutting system positioned adjacent to the patient support on a first lateral side of the patient support comprising a base, at least one fixed cutter tracking array, a powered articulated arm coupled to the base, an end effector coupled to an end arm segment of the articulated arm, and a cutting tool coupled by the end effector defining a cutting edge; an electronic user controller control comprising a plurality of manually-operable control inputs that are configured to control operational parameters of the cutting tool, the electronic controller positioned adjacent to the patient support on a second lateral side of the patient support that opposes the first lateral side of the patient support; at least one fixed patient tracking array configured to couple to the patient at a fixed positional relationship to a surgical site of the patient; and a navigation system positioned on the first lateral side of the patient support, comprising a base, at least one tracking camera, at least one graphical display, and a computer tracking system configured to determine the three-dimensional position of the cutting edge via the at least one tracking camera based on at least one of movement of the articulated arm, the at least one fixed cutter tracking array, and the relative at least one fixed patient tracking array.

In some embodiments, the system is configured such that the at least one fixed cutter tracking array is positioned distally of the base and the articulated arm of the active cutting system. In some such embodiments, the system is configured such that the at least one fixed cutter tracking array is positioned within a line of sight between the at least one camera and the surgical site as positioned by the patient support. In some such embodiments, the at least one fixed cutter tracking array comprises a plurality of identifiably-shaped portions at predefined relative positions and orientations with respect to each other.

In some embodiments, the system is configured such that the at least one fixed cutter tracking array is positioned along a linear line of sight between the at least one camera and the surgical site as positioned by the patient support.

In some embodiments, the at least one fixed patient tracking array comprises a plurality of identifiably-shaped portions at predefined registered relative positions and orientations with respect to the surgical site when affixed thereto. In some such embodiments, the at least one fixed patient tracking array comprises a first fixed patient tracking array configured to affix to a first anatomical structure related to the surgical site, and a second fixed patient tracking array configured to affix to a second anatomical structure related to the surgical site that differs from the first anatomical structure. In some such embodiments, the first anatomical structure is a distal femur, and the second anatomical structure surgical site is a proximal tibia.

In some embodiments, the surgical site is a portion of an articular surface of a tibia and/or an articular surface femur of a knee joint. In some embodiments, the at least one fixed patient tracking array faces laterally toward the first lateral side and distally.

In some embodiments, the system is configured such that the surgical site is positioned distally of the user controller and proximally of the navigation system. In some such embodiments, the system is configured such that the surgical site is positioned proximate to the second lateral side of the patient support and distal to the first lateral side of the patient support. In some such embodiments, the system is configured such that the articulated arm extends over the patient and the patient support to proximate to the surgical site from the first lateral side of the patient support laterally to the surgical site.

In some embodiments, the system is configured such that the surgical site is positioned proximate to the second lateral side of the patient support and distal to the first lateral side of the patient support. In some embodiments, the system is configured such that the articulated arm extends over the patient and the patient support to proximate to the surgical site from the first lateral side of the patient support laterally to the surgical site.

In some embodiments, a base arm segment of the articulated arm is coupled to the base of the active cutting system at an angle that extends laterally toward the second lateral side of the patient support as it extends upwardly from the base. In some embodiments, the system is configured such that the navigation system is positioned on the first lateral side of the patient support distally past a distal end of the patient support. In some embodiments, the system is configured such that the user controller and the base of the active cutting system are substantially aligned in the proximal-distal direction.

In some embodiments, the system is configured such that: the at least one fixed cutter tracking array is positioned distally of the base and the articulated arm of the active cutting system; the at least one fixed cutter tracking array is positioned within a line of sight between the at least one camera and the surgical site as positioned by the patient support; the surgical site is positioned distally of the user controller and proximally of the navigation system; the surgical site is positioned proximate to the second lateral side of the patient support and distal to the first lateral side of the patient support; and the articulated arm extends over the patient and the patient support to proximate to the surgical site from the first lateral side of the patient support laterally to the surgical site. In some such embodiments, the system is further configured such that the navigation system is positioned on the first lateral side of the patient support distally past a distal end of the patient support. In some such embodiments, the system is further configured such that the user controller and the base of the active cutting system are substantially aligned in the proximal-distal direction.

In some embodiments, the base of the active cutting system is a movable cart. In some embodiments, the base of the navigation system is a movable cart. In some embodiments, the system is configured such that the plurality of control inputs control the operation of the cutting tool while executing at least one programmed cut path but do not alter the configuration of the at least one programmed cut path.

In some embodiments, the robotic system is configured to autonomously adjust the relative orientation of the arm segments of the articulated arm to autonomously spatially translate the cutting tool and cut material at the surgical site along at least one programmed cut path. In some such embodiments, the system is configured such that the plurality of control inputs control the operation of the cutting tool while executing the at least one programmed cut path but do not alter the configuration of the at least one programmed cut path.

In some embodiments, the navigation system is configured to display at least one of a representation of the cutting tool and the surgical site based on the actual positions thereof. In some embodiments, the active cutting system further comprises at least one graphical display positioned at a back lateral side of the active cutting system that faces laterally away from the patient support, and wherein the active cutting system is configured to display a representation of at least one of at least one preprogrammed cut path and the position of the cutting tool relative to the at least one preprogrammed cut path and/or the surgical sight.

In some embodiments, the cutting edge of the cutting tool is configured to cut while being translated along a cutting pathway, and wherein the end effector comprises a powered drive portion that translates the cutting tool along the cutting pathway. In some embodiments, the navigation system is configured to determine and track the three-dimensional position of the cutting edge in consideration of a plurality of registration movements of the articulated arm and the position and/or orientation of the cutting edge relative to that of the at least one tracking camera. In some embodiments, the navigation system is configured to determine and track the three-dimensional position of the cutting edge relative to the surgical site via the at least one tracking camera based on movement of the articulated arm and the relative positions and orientations of the at least one fixed cutter tracking array and the at least one fixed patient tracking array.

In some embodiments, the plurality of control inputs comprises at least one first control input that, when selectively actuated by a user, is configured to direct the robotic system to advance or retract the cutting tool along a current programmed cut path that the robotic system is executing. In some such embodiments, the plurality of control inputs comprises at least one second control input that, when selectively actuated by a user, is configured to direct the robotic system to translate the cutting tool along a current programmed cut path that the robotic system is executing at an increased or decreased velocity as compared to a current programmed velocity. In some such embodiments, the he plurality of control inputs comprises at least one control input that, when selectively actuated by a user, is configured to stop the end effector from translating the cutting tool along the cutting pathway. In some such embodiments, the controller is configured as a foot controller such that the plurality of control inputs are configured to be selectively actuated by a user's foot.

In some embodiments, the controller is configured as a foot controller such that the plurality of control inputs are configured to be selectively actuated by a user's foot. In some such embodiments, the foot controller comprises a foot pedal that is configured to be selectively engaged by the underside of a user's foot and a housing positioned adjacent to at least one side of the foot pedal, and wherein the foot pedal is configured such that the user can articulate the foot pedal between a first forefoot position with a forefoot portion of the foot pedal being depressed, and a second hindfoot position with a hindfoot portion of the foot pedal being depressed. In some embodiments, the first forefoot position of the foot pedal actuates a first control input that is configured to direct the robotic system to advance the cutting tool along a current programmed cut path that the robotic system is executing when selectively actuated by a user. In some embodiments, the second hindfoot position of the foot pedal actuates a second control input second control input that is configured to direct the robotic system to retreat the cutting tool along a current programmed cut path that the robotic system executed immediately previous thereto when selectively actuated by a user. In some embodiments, the housing comprises a plurality of the plurality of control inputs that are configured to be selectively engaged by the user's foot.

In some embodiments, the cutting tool is a sagittal cutting blade with cutting teeth positioned at a longitudinal end thereof, the sagittal cutting blade being configured to cut when translated longitudinally and oscillated along a cutting pathway that lies in a plane in which the blade is aligned about an axis of oscillation. In some such embodiments, the cutting pathway is at least one programmed cut path that extends through a first bone of the patient such that the cutting tool cuts the first bone when executing the least one programmed cut path. In some such embodiments, the robotic system is configured to autonomously adjust the relative orientation of the arm segments of the articulated arm to autonomously spatially translate the cutting tool along the least one programmed cut path. In some such embodiments, the system is configured such that the plurality of control inputs control the operation of the cutting tool while executing the at least one programmed cut path but do not alter the configuration of the at least one programmed cut path.

In some embodiments, the patient support comprises a fixation assembly that is configured to fix the position and/or orientation of the surgical site of the patient relative to the patient support.

In another aspect, the present disclosure provides method of cutting material at a surgical site, comprising utilizing the system according to any of surgical systems disclosed above to autonomously translate the cutting tool along a least one programmed cut path to cut material at the surgical site.

It should be appreciated that all combinations of the foregoing aspects and additional concepts discussed in greater detail below (provided such concepts are not mutually inconsistent) are contemplated as being part of the inventive subject matter and to achieve the advantages disclosed herein.

These and other objects, features and advantages of this disclosure will become apparent from the following detailed description of the various aspects of the disclosure taken in conjunction with the accompanying drawings.

### BRIEF DESCRIPTION OF THE DRAWINGS

The accompanying drawings, which are incorporated in and constitute a part of the specification, illustrate embodiments of the disclosure and together with the detailed description herein, serve to explain the principles of the disclosure. It is emphasized that, in accordance with the standard practice in the industry, various features are not drawn to scale. In fact, the dimensions of the various features may be arbitrarily increased or reduced for clarity of discussion. The drawings are only for purposes of illustrating preferred embodiments and are not to be construed as limiting the disclosure.

FIG. 1 illustrates, in one example, a top view of a surgical robotic system layout in a surgical theater, in accordance with one or more aspects of the present disclosure;

FIG. 2 illustrates, in one example, an elevational perspective view of the surgical robotic system layout of FIG. 1, in accordance with one or more aspects of the present disclosure;

FIG. 3 illustrates, in one example, a first lateral side perspective view of the surgical robotic system layout of FIG. 1, in accordance with one or more aspects of the present disclosure;

FIG. 4 illustrates, in one example, a second lateral side perspective view of the surgical robotic system layout of FIG. 1, in accordance with one or more aspects of the present disclosure;

FIG. 5 illustrates, in one example, an elevational distal perspective view of the surgical robotic system layout of FIG. 1, in accordance with one or more aspects of the present disclosure;

FIG. 6 illustrates, in one example, a medial side perspective view of the surgical robotic system layout of FIG. 1, in accordance with one or more aspects of the present disclosure; and

FIG. 7 illustrates, in one example, a graphic representation of a computer system and associated devices to incorporate and/or use aspects described herein, in accordance with one or more aspects of the present disclosure.

### DETAILED DESCRIPTION FOR CARRYING OUT THE INVENTION

Aspects of the present disclosure and certain examples, features, advantages, and details thereof, are explained more fully below with reference to the non-limiting examples illustrated in the accompanying drawings. Descriptions of well-known materials, fabrication tools, processing techniques, etc., are omitted so as not to unnecessarily obscure the relevant details. It should be understood, however, that the detailed description and the specific examples, while indicating aspects of the disclosure, are given by way of illustration only, and are not by way of limitation. Various substitutions, modifications, additions, and/or arrangements, within the spirit and/or scope of the underlying inventive concepts will be apparent to those skilled in the art from this disclosure.

Approximating language, as used herein throughout disclosure, may be applied to modify any quantitative representation that could permissibly vary without resulting in a change in the basic function to which it is related. Accordingly, a value modified by a term or terms, such as “about” or “substantially,” is not limited to the precise value specified. For example, these terms can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%. In some instances, the approximating language may correspond to the precision of an instrument for measuring the value. Any examples of operating or configuration parameters are not exclusive of other parameters of the disclosed embodiments.

Terminology used herein is for the purpose of describing particular examples only and is not intended to be limiting. As used herein, the singular forms “a”, “an” and “the” are intended to include the plural forms as well, unless the context clearly indicates otherwise. Furthermore, references to “one example” are not intended to be interpreted as excluding the existence of additional examples that also incorporate the recited features. Moreover, unless explicitly stated to the contrary, the terms “comprising” (and any form of “comprise,” such as “comprises” and “comprising”), “have” (and any form of “have,” such as “has” and “having”), “include” (and any form of “include,” such as “includes” and “including”), and “contain” (and any form of “contain,” such as “contains” and “containing”) are used as open-ended linking verbs. As a result, any examples that “comprises,” “has,” “includes” or “contains” one or more step or element possesses such one or more step or element, but is not limited to possessing only such one or more step or element.

As used herein, the terms “may” and “may be” indicate a possibility of an occurrence within a set of circumstances; a possession of a specified property, characteristic or function; and/or qualify another verb by expressing one or more of an ability, capability, or possibility associated with the qualified verb. Accordingly, usage of “may” and “may be” indicates that a modified term is apparently appropriate, capable, or suitable for an indicated capacity, function, or usage, while taking into account that in some circumstances the modified term may sometimes not be appropriate, capable or suitable. For example, in some circumstances, an event or capacity can be expected, while in other circumstances the event or capacity cannot occur—this distinction is captured by the terms “may” and “may be.”

The term “coupled” and like terms are used herein to refer to both direct and indirect connections. As used herein and unless otherwise indicated, the term “entirety” (and any other form of “entire”) means at least a substantial portion, such as at least 95% or at least 99%. The term “entirety” (and any other form of “entire”), as used herein, is thereby not limited to 100%, unless otherwise indicated. As used herein, the term “layer”

In this detailed description and the following claims, the words proximal, distal, anterior, posterior, medial, lateral, superior and inferior are defined by their standard usage for indicating directional or positional reference based on reference to the human body. For example, “proximal” means the portion of a device or body part nearest the torso, while “distal” indicates the portion of the device or body part farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure. Further, the terms “proximal” and “distal” are used similar to the terms “superior” and “inferior” in that “proximal” is used to refer to a direction or portion that is closest or closer to the head of a patient, and “distal” is used to refer to a direction or portion that is closest or closer to the feet of a patient. The terms “proximal” and “distal” are also used as relative distance references in that “proximal” is used to refer to a feature that is closer to a reference point than a “distal” reference point.

Similarly, positions or directions may be used herein with reference to anatomical structures or surfaces. For example, as the devices, features, systems and methods are described herein with reference to use with the bones of a knee, the bones of the upper leg and lower leg (e.g., tibia and femur) may be used to describe the surfaces, positions, directions or orientations of the devices, systems, features and methods. Further, the devices, systems and methods, and the aspects, components, features and the like thereof, disclosed herein are described with respect or reference to one side of the body for brevity purposes. However, as the human body is relatively symmetrical or mirrored about a line of symmetry (midline), it is hereby expressly contemplated that the devices, systems, features and methods, and the aspects, components, features and the like thereof, described and/or illustrated herein may be changed, varied, modified, reconfigured or otherwise altered for use or association with another side of the body for a same or similar purpose without departing from the spirit and scope of the invention. For example, the devices, systems, features and methods, and the aspects, components, features and the like thereof, described herein with respect to the left knee or a first lateral side of the patient may be mirrored so that they likewise function with the opposite right knee or the second lateral side of the patient, and vice versa. Further, the devices, systems, features and methods, and the aspects, components, features and the like thereof, disclosed herein are described with respect to a knee for brevity purposes, but it should be understood that the devices, systems, features and methods may be used with other bones of the body, for example a lower extremity or upper extremity, such as any bone or bones of a patient.

Components, aspects, features, configurations, arrangements, uses and the like described, illustrated or otherwise disclosed herein with respect to any particular embodiment may similarly be applied to any other embodiment disclosed herein.

As described further below, surgical robots, surgical robotic systems and related surgical methods with unexpectedly advantageous component layouts are disclosed. The surgical robotic systems include a controller that allows a user to electronically control parameters of movement of a cutting tool along a prescribed or determined cut path. The surgical robotic systems and methods provide for user inputs to an active or autonomous robotic system to control parameters of movement of a cutting tool along a prescribed or determined cut path. The surgical robotic systems and related surgical methods thus comprise an electronic controller that provides intuitive user control of an active robot along a prescribed or determined cut path thereof.

The surgical robotic systems and related surgical methods further include a robotic cutting system with a robotic articulated arm, and end effector and a cutting tool configured to cut when moved by the end effector along a cutting pathway (e.g., reciprocating planar pathway or rotational pathway, for example). The surgical robotic systems and related surgical methods also include a navigation system with at least one tracking camera and at least one display screen that displays a representation of the surgical site (e.g., bones) in their current position and/or orientation, an implant superimposed on the surgical site, cut paths or areas to accommodate the implant, previous cut paths/area, a current cut path/area, future cut paths/areas, or other information related to the position, orientation and/or movement of the cutting tool and/or the surgical site (such as relative to each other). The navigation system may thereby track and display parameter of a surgical cutting operation.

The present disclosure is generally directed to surgical robots, surgical robotic systems and related surgical methods, and in particular orthopedic surgical robots, orthopedic surgical robotic systems and related orthopedic surgical methods, with the user controller, robotic cutting system and navigation system that are arranged in a surgical theater in a layout that unexpectedly provides advantageous usability and enhanced performance of the system and the theater. The term “surgical theater” is used to reference the physical space, such as an operating room, operating suite, or operation suite, which may or may be in a medical facility (e.g., a hospital) where a surgical operation or procedure, and in particular an orthopedic surgical procedure, such as a total or partial knee arthroplasty, is carried out. The physical space may be an aseptic environment or the like.

The surgical robotic systems include a robotic cutting system and a navigation system, that that monitors/tracks and controls the movement of a cutting tool of the robotic cutting system and monitors/tracks the surgical site, (and potentially the user controller and/or a patient tracking array(s)) situated relative to a patient that unexpectedly advantageously minimizes occlusion by users of one or more tracking camera of the navigation system. The layout of the robotic cutting system and the navigation system (and potentially the user controller and/or a patient tracking array(s)) relative to the patient also unexpectedly advantageously improves tracking and cutting tool positioning accuracy. The layout of the robotic cutting system and a navigation system (and potentially the user controller and/or a patient tracking array(s)) relative to the patient also unexpectedly advantageously minimizes the likelihood of the robotic arm from colliding with a user or the patient. The layout of the robotic cutting system and a navigation system (and potentially the user controller and/or a patient tracking array(s)) relative to the patient also unexpectedly advantageously improves the accessibility and breadth of the user's workspace area. The layout of the robotic cutting system and a navigation system (and potentially the user controller and/or a patient tracking array(s)) relative to the patient also unexpectedly advantageously reduces the likelihood and/or volume of splatter from a cutting operation onto tracking areas (e.g., the patient tracking array(s) and/or at least one cutter tracking array) that are utilized by the navigational system to determine the position/orientation of the cutting tool and/or a surgical site of the patient. The layout of the robotic cutting system and a navigation system relative (and potentially the user controller and/or a patient tracking array(s)) to the patient also unexpectedly advantageously allows a user of the robotic cutting system to easily access and reposition one or more display and/or camera of the navigation system. The layout of the robotic cutting system and a navigation system (and potentially the user controller and/or a patient tracking array(s)) relative to the patient also unexpectedly advantageously provides an unobstructed view path of one or more displays of the navigation system in line with a user's line of sight when the user faces the surgical site.

In some embodiments, the electronic controller of the surgical robotic systems is configured as a hands-free device such that it is configured to be utilized via a foot of a user. The electronic controller of the surgical robotic systems is configured to allow a user to provide or enter inputs from a user that command the robot to alter parameters of movement of the cutting tool along a determined, prescribed or programmed cut path. The electronic controller may feature tactile control inputs that a user can physically engage or activate to correspondingly control a parameter of the movement of the cutting tool along a determined, prescribed or programmed cut path of the cutting tool that is currently being executed by the robotic system. In some embodiments, the tactile control inputs may comprise electrical switches, which may provide tactile or physical feedback/movement. In some such embodiments, the electrical switches are configured as depressible buttons, rocker switches, a foot pedal or a combination thereof.

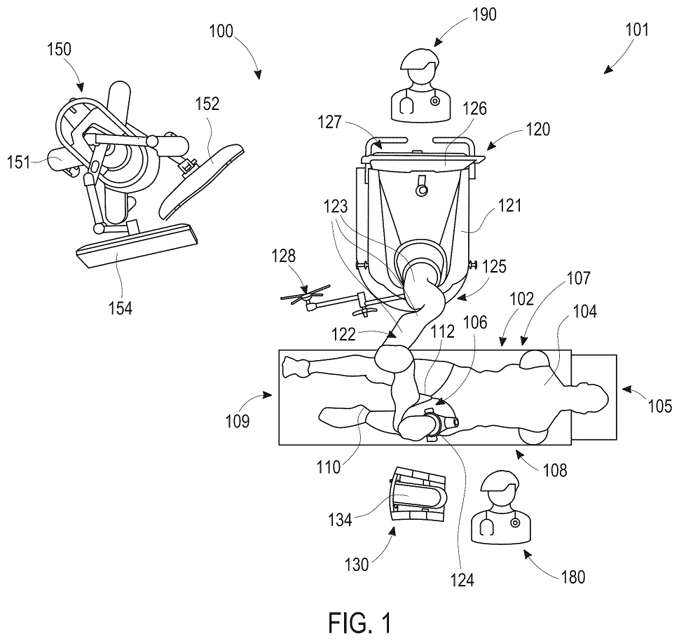

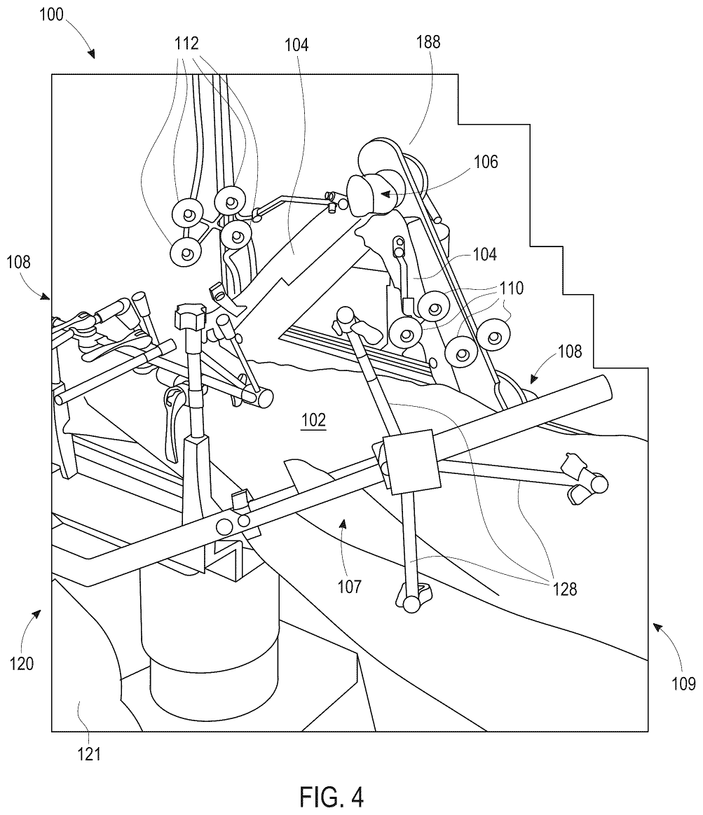

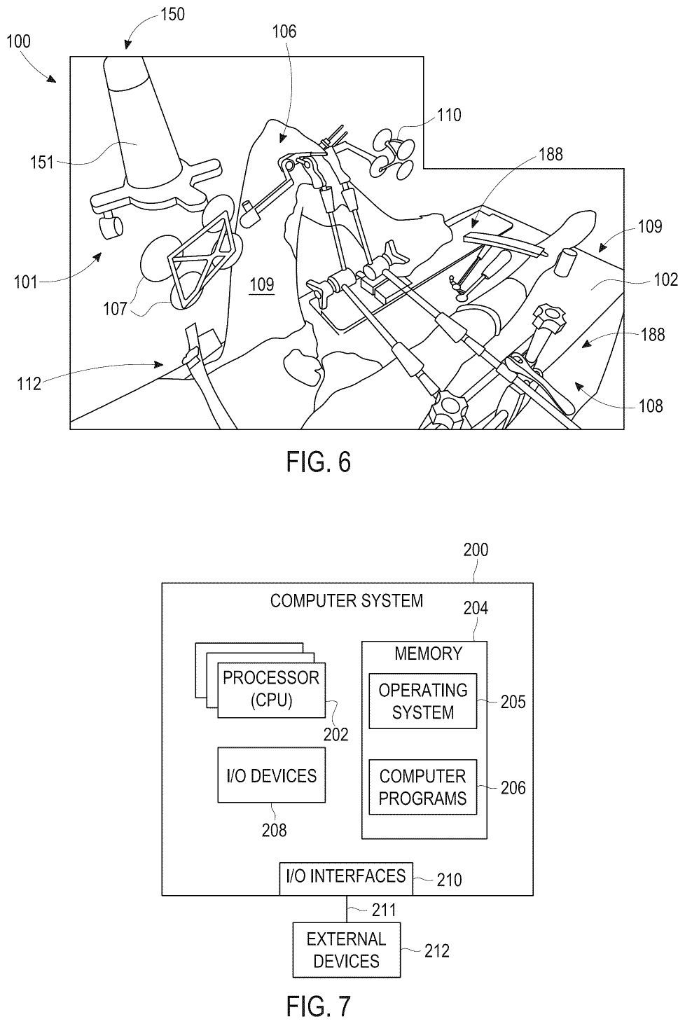

An exemplary orthopedic robotic surgical system, and layout thereof, is illustrated in FIGS. 1-6, and referenced generally by reference numeral 100. As shown in FIGS. 1-6, the orthopedic robotic surgical system 100 according to the present disclosure may include, inter alia, a robotic cutting system 120, a navigation system 150, and potentially a user controller 130, in a surgical theater 101. As shown, the robotic cutting system 120 may include a base 121, an articulated robotic arm 122, an end effector 124 and a cutting tool or device 129. As shown in FIGS. 1-5, the at least one fixed cutter tracking array 128 can be positioned distally of the base 121 and the articulated arm 122 of the active cutting system 120 to prevent occlusion thereof from the camera system 152 of the navigation system 150.

As depicted, the robotic cutting system 120 may be configured as a surgical robot. For example, the robotic cutting system 120 may be biocompatible, and configured be sterilized to such a degree, as required in surgical settings. However, in other embodiments, the robotic cutting system 120 may be configured as an industrial or other non-surgical robotic devices or systems. The term “surgical robot” as used herein in reference to the exemplary illustrative robot/robotic system embodiments, and is not meant in a limiting sense, and any and all description herein directed to a “surgical robot” or the like equally applies to a generic robot/robotic system or an industrial or other non-surgical robot/robotic system.

It is noted that robotic system 100, and component thereof (such as the navigational system 150 and the robotic cutting system 120) may comprise or be operably connected to a computer system (e.g., memory, processor, etc.), as shown in FIG. 7 for example, that controls movement of the cutting tool along one or more determined, prescribed or programmed cut paths, via movement of the articulated arm for example, potentially operation of the end effector, and the operation of the tracking system and the cutting system otherwise. The tracking system may track a surgical site 106 (e.g., bone or bone portions thereof, such as of a joint) such that its spatial position and/or orientation is monitored, and a representation thereof (e.g., a three-dimensional model thereof) is displayed on a display screen of the tracking system (in real time). It is noted that the surgical site 106 may be tracked via at least one tracking camera of the tracking system and at least one fixed patient tracking array coupled to the patient at a fixed positional relationship to the surgical site 106, which is pre-registered by the tracking system. Accordingly, the tracking system is able to monitor the three-dimensional position and/or orientation of the surgical site 106, via the tracking camera, based on movements of the at least one fixed patient tracking array, and display a corresponding representation of the surgical site 106 to the user on a display of the tracking system (and/or another display). Further, the tracking system may superimpose or display an implant or other surgical construct on/at the surgical site 106 (and the engagement surfaces thereof, and thus required cut surfaces) that is registered to the surgical site 106, so that a user can assess fitment, positioning and orientation thereof virtually.

For example, in some embodiments, the robotic system 100 may comprise one or more control unit with programming, and potentially a user interface (UI), as shown in FIGS. 1-3, such with as the tracking system and/or the cutting system. The control unit may include at least one processing circuit, at least one input/output device, and at least one storage device or memory having at least one database or cutting instructions stored therein. The control unit may have a control algorithm or programming code for controlling the position of the cutting tool 129 (such as via the joint angle between segments of the articulated arm, for example) and/or tracking and displaying representations of the surgical site 106. The control algorithm or programming code may be a default control algorithm or include inputs from, for example, the UI and/or another interface.

As shown in FIGS. 1-4, the articulated arm 122 may extend from the base 121 and include a plurality of rigid arm or body segments/parts 123, and a plurality of joints that connect adjacent segments 123 (and a first or base segment to the base). The plurality of joints may include, for example, four, five or six individual segments 123 that are coupled together via three, four or five joints, respectively. In some other embodiments, the articulated arm 122 may include at least two segments 123 and at least one joint coupling the at least two segments 123 together, or more than six segments 123 and more than five joints coupling the segments 123 together.

Each arm segment of the articulated arm 122 may define an axial axis extending along its longitudinal length. The joints may be configured such that the arm segments 123 can rotate about their axes and/or articulate angularly with respect to each other such that the axes of adjacent segments 123 are angularly offset. In some embodiments, one or more of the joints may be configured to allow multiple degrees of freedom between adjacent arm segments 123 (and, potentially, the base segment and the base 121). In some such embodiments, at least one of the joints may be configured to provide six degrees of freedom.

The articulated arm 122 may further comprise motors, actuators or other adjustment devices that are configured to adjust the axial rotation and/or angular orientation between adjacent segments. In this way, the active robotic cutting system 120 can control the arrangement of the articulated arm 122 to translate the cutting tool 129 three-dimensionally in space and relative to a workpiece (e.g., a patient) to, ultimately, cut one or more portions of the workpiece. As noted above, the active robotic cutting system 120 may include control software that dictates or instructs, inter alai, the articulated arm 122 of the active robotic cutting system 120 to adjust in particular ways (i.e., adjustment of the joints) to accomplish determined, prescribed or programmed cut path movements of the cutting tool 129. Stated differently, the active robotic cutting system 120 may include control software that dictates or instructs, inter alai, the articulated arm 122 of the active robotic cutting system 120 to adjust in particular ways (i.e., adjustment of the joints) to translate the cutting tool 129 in three-dimensional space along one or more (e.g., a series of a plurality of) determined, prescribed or programmed cut paths of the cutting tool 129 to cut the workpiece along or according to the determined, prescribed or programmed cut paths. The paths of the cutting tool 129 may thereby be predetermined, pre-prescribed or pre-programmed in the active robotic cutting system 120 such that the user does not dictate the cut paths of the cutting tool 129 that the cutting tool 129 travels along to cut the workpiece. As explained further below, the user may be provided with control over parameters or metrics of the movement of the cutting tool 129 along the cut paths via a controller, but not the configuration or three-dimensional parameters of the cut paths themselves.

The base 121 of the surgical active robotic cutting system 120 may be formed as, or fixed to, a movable cart, or to the ground or base structure, such that the base 121 provides a fixed frame of reference for defining the position, orientation, and motion of the plurality of joints and the plurality of arm segments 123 relative to the base 121. The base 121 may be used to define a frame of reference, such as, for example, a set of three-dimensional axes (e.g., x, y, z), which may be used to define positions, orientations, and motions of the surgical active robotic cutting system 120 and of objects relative to the surgical active robotic cutting system 120.

For example, as shown in FIGS. 1-5, the active robotic cutting system 120 may include an at least one fixed cutter tracking array 128 with fiducials or identifiable shaped and/or colored portions of a known relative position and/or orientation which a camera or other detection system 152 of the navigational system 150 can detect (visually) and utilize to determine the position and/or orientation of the active robotic cutting system 120, such as the base 121 and/or the first arm segment thereof. The fixed cutter tracking array 128 thereby includes a plurality of identifiably-shaped and/or colored portions at predefined relative positions and orientations with respect to each other.

It is noted that the camera or other detection system 152 of the navigational system 150 may also determine the position and/or orientation of the cutting tool 129 (e.g., the tip thereof) directly/independently and/or based on the position and/or orientation and the configuration of the arm 122 or movements thereof, such as after registration thereof. A frame of reference defined relative to the base 121 via the at least one fixed cutter tracking array 128 may also be known as a world frame, a base, a base frame, a frame, or a tool frame. It is noted that with the position and/or orientation of an object defined or calculated in relation to the fixed frame of reference, the object may also be defined in the same frame of reference as the active robotic cutting system 120, and the navigational system 150 may calculate the position and/or orientation of the object. As such, the surgical active robotic cutting system 120 may programmably interact with the defined objects, positions, and/or orientations.

For example, in some embodiments, the navigational system 150 may utilize the camera system 152 (and/or a tracking tool) to track/register the relative positions and orientations of the at least one fixed cutter tracking array 128 and the cutting edge of a cutting tool (e.g., via a reference or registration tool that replicates the cutting edge of the actual cutting tool 129). The robotic cutting system 120 may perform a series or plurality of movements of the articulated arm 122, and register the position and orientation of the cutting edge relative to the at least one fixed cutter tracking array 128. Through the series of movements and registrations, the system 100 may compile sufficient articulated arm 122 reference movement parameters and corresponding cutting locations/orientations that can be used by the robotic cutting system 120 to adjust the articulated arm 122 to effectuate a particular movement or three-dimensional position/orientation of the cutting edge. In this way, based on the position and orientation of the at least one fixed cutter tracking array 128, determined by the navigational system 150, the system 100 can determine articulated arm 122 movement parameters that position/orient the cutting edge at a particular location/orientation in space.

As shown in FIGS. 1-3, the active robotic cutting system 120 may include an end effector 124 coupled (e.g., rotatably coupled) to an end, last or termination arm segment of the articulated arm 122, such as via a rotatable connector assembly therebetween. The rotatable connector assembly may be configured such that the end effector 124 is rotatable about a longitudinal axis of the end arm segment. As shown in FIGS. 1-3, the end effector 124 may be configured such that the axis of the end arm segment and the axis of the cutting tool 129, along which the cutting direction of the cutting tool 129 extends, are angled with respect to each other. In the illustrated exemplary embodiment, the axis of the cutting tool 129 (i.e., the direction the cutting tool 129 is configured to cut) and the axis of the end arm segment are oriented perpendicular (or normal) to each other.

Referring further to FIGS. 1-3, since the position, orientation, and motion of the plurality of joints and the plurality of arm segments 123 relative to the base 121/at least one fixed cutter tracking array 128 may be defined, and the angular orientation of the end effector 124 with respect to the end arm segment of the articulated arm 122, the position and/or orientation of the cutting tool 129 extending from the end effector 124 can be calculated or determined by the robotic system 100, or vice versa. In another embodiment, at least one fixed cutter tracking array 128 may be utilized to determine/detect the position and/or orientation of the base 121/at least one fixed cutter tracking array 128 via the camera or other detection system 150 of the navigational system 150, as described above, and/or the position and/or orientation of the cutting tool 129 (e.g., a cutting portion or tip thereof) may be determined/detected via the camera or other detection system 150. It is understood that an exemplary illustrative cutting tool 129 may be configured for cutting bone or other tissue, however the cutting tool 129 may be replaceable with a different cutting tool or a non-cutting implement that may function as, for example, a marking device, registration device, or a viewing device.

The cutting tool 129 may include an attachment, tang or hub portion at a proximal end portion that couples with an attachment mechanism of the end effector 124. For example, the end effector 124 may include a chuck or other attachment mechanism configured to mate with the attachment portion of the cutting tool 129, and removably secure the cutting tool 129 and the end effector 124 together. The end effector 124 may thereby be capable of physically translating or moving the cutting tool 129 along a cutting pathway along which the cutting tool 129 is configured to cut. The axis of the cutting tool 129 may extend through the attachment portion and the end effector 124, and in some embodiment, the cutting tool/blade 129 (and the end effector 124) may be longitudinally extended along the axis.

In some embodiments, the cutting tool 129 configured as an axially extending rotary cutting tool. The cutting tool 129 may be configured to cut along a cutting pathway that extends about the longitudinal axis of the cutting tool 129 (i.e., is configured to cut when the cutting tool 129 is rotated about its axis and translate axially/longitudinally through a workpiece). The end effector 124 may thereby be configured to provide such rotation or torque to the cutting tool 129 such that it is rotated along the cutting pathway.

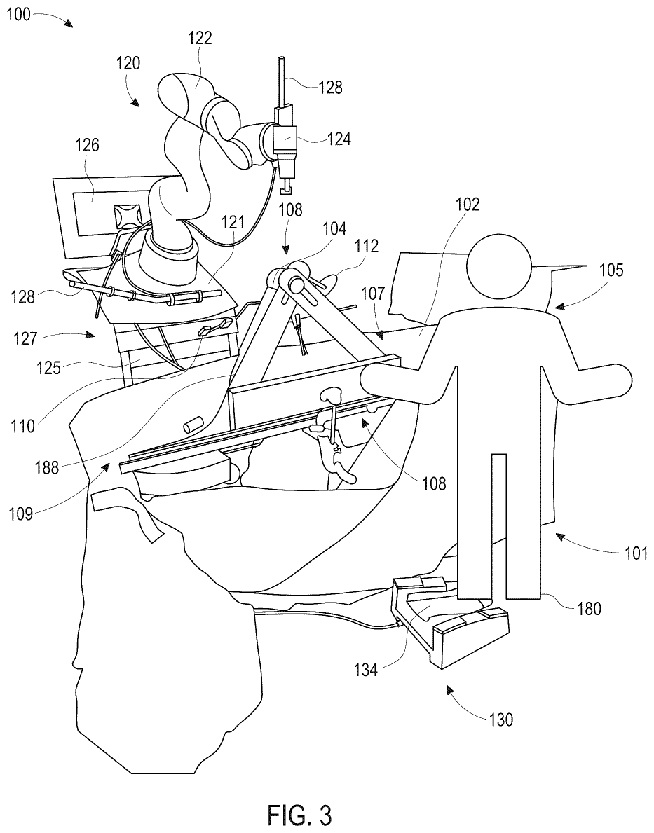

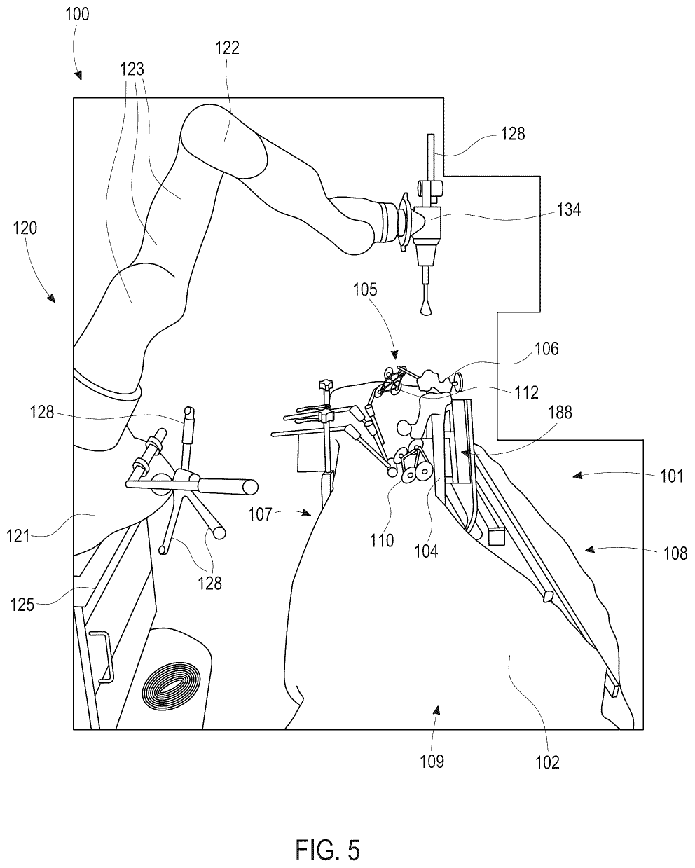

As shown in FIGS. 3 and 5, in some other embodiments, the cutting tool 129 is configured as a cutting blade. For example, the cutting tool 129 may be a saw blade that has a thin, flat, elongated shape with a cutting edge at a distal tip or end portion of a blade body portion. The thin, flat design may minimize the size of the blade's kerf and allow the blade to make an accurate, straight cut. The cutting edge may be generally oriented along a direction that is orthogonal to the direction of blade elongation and contains a plurality of teeth and/or abrasives. Thus, when the blade 129 is translated along a cut pathway, the cutting edge can be translated axially/longitudinally against and through a workpiece (e.g., a bone or other tissue that requires resection) as it is translated along its cutting pathway or direction.

The cutting blade 129 (e.g., at least the cutting edge thereof) may be configured to cut when moved/translated in the cutting pathway, such as in a reciprocating motion (along forward and/or back strokes), along a linear direction (colinear with the cutting edge), along a plane (e.g., two dimensions) or in a three-dimensional pattern. The exemplary illustrative cutting blade is configured to be pivoted back and forth, or oscillated, in the cutting pathway which extends along the plane in which the cutting blade is oriented and is orthogonal to the direction of blade elongation. The cutting blade of may be designed such that the cutting direction or pathway oscillates linearly laterally or in an arc extended along the plane of the blade. The blade may thereby be configured as a sagittal saw blade.

In some embodiments, the end effector 124 of the robotic cutting system 120 may be configured to oscillate (i.e., translate in a back and forth manner) the cutting tool 129, which is configured as a cutting blade, along the oscillatory cutting direction or pathway. As explained above, the cutting tool 129 may be configured as a sagittal cutting blade, and the oscillatory cutting pathway may extend along a plane defined by the blade. When the cutting tool 129 is configured as a sagittal saw blade, the blade performs a cutting action by being translated in a cut path extending along the longitudinal axis (e.g., via the articulated arm 122, at least in part) (i.e., longitudinally/axially) in a direction extending from the coupling portion to the tip portion thereof as the blade (and the cutting teeth thereof) is being oscillated along the oscillatory cutting pathway (i.e., in cutting strokes). Because of the motion along the oscillatory cutting pathway applied by the end effector 124, and the forward pressure applied by the robotic cutting system 120 (e.g., via the articulated arm 122, at least in part) along a cut path, the teeth of the cutting blade 129 cut and separate material of the surgical site 106 (e.g., tissue, such as bone tissue).

As shown in FIGS. 1-3, in some embodiments, the base 121 coupled to the first segment of the articulating arm 122 may be a movable cart. The cart base 121 may thereby provide a movable base for the robotic cutting system 120 to make the robotic cutting system 120 a compact transportable or a movable cutting robot construct. The robotic cutting system 120 may also include a screen or graphical user interface 126 that allows user to see and/or visualize the operation of the robotic cutting system 120. For example, the robotic cutting system 120 may be configured to display the status of the operation of the cutting tool 129 (e.g., via the operation of the end effector 124), the position and/or orientation of the cutting tool 129 (potentially relative to a patient or other workpiece), and/or the status of one or more cut paths or cutting operations or cutting parameters/metrics. The screen or graphical user interface 126 may also allow a user to operate robotic cutting system 120 (e.g., alter a metric or characteristic of the operation of the robotic cutting system 120). In some embodiments, the base of the articulating arm 122, and thereby at least the base portion of the articulating arm 122, may be positioned on a lateral side or end 125 of the base 121/robotic cutting system 120, and the screen 126 may be positioned on an opposing lateral end or side 127 of the base 121/robotic cutting system 120. As explained further below, the base portion of the articulated arm 123 may be angled with respect to vertical such that the articulating arm 122 is biased off the respective front lateral side or end 125 of the base 121/robotic cutting system 120 and away from a user at the rear/back end or side 127 of the base 121/robotic cutting system 120 with the screen or graphical user interface 126. As shown in FIG. 1, a user 190 may thereby be positioned adjacent to the rear/back end or side 127 of the base 121/robotic cutting system 120 so as to view the display/screen 126. The graphical display 126 may thereby be positioned at a back lateral side 127 of the active cutting system 120, and face laterally away from the articulated arm 122 and a patient support 102 (and patient 104 thereon).

It is noted that the first arm segment of the articulated arm 122 is mounted proximate to the respective front lateral side/end 125 of the cart or base 121, and the arm 122 is thereby cantilevered over such respective lateral side/end 125 of the cart or base 121. For example, in some embodiments, the first arm segment may define an axis that is angled within the range of about 15 degrees to about 55 degrees from vertical, or within the range of about 25 degrees to about 25 degrees from vertical. In the exemplary illustrated embodiment, the axis of the first arm segment is angled about 35 degrees from vertical.

As shown in FIGS. 1-3, the base 121/robotic cutting system 120 is positioned within the surgical theater 101 laterally adjacent to a first lateral side 107 of a patient support device or construct 102. The patient support 102 is configured to securely or stably physically support at least a portion of a patient 104. The patient support 102 may be a table, platform, seat or any other structure that stably physically support the patient 104 such that the patient 104 is provided at a fixed location within the surgical theater 101. As shown in FIG. 1, the patient support 102 defines or includes the first lateral side 107, a second lateral side 108 that medially-laterally opposes the first lateral side 107, a proximal end 105, and a distal end 109 that proximally-distally opposes the proximal end 105. The patient 104 may likewise be situated on the patient support 102 in a corresponding orientation, as shown in FIG. 1. As shown in FIG. 1, the system 100 and the patient support 102 and the patient 104 may be configured such that the surgical site 106 is positioned proximate to the second lateral side 108 of the patient support 102 and distal to the first lateral side 107 of the patient support 102. As noted above, a base arm segment of the articulated arm 122 may be coupled to the base 121 of the active cutting system 120 at an angle that extends laterally toward the second lateral side 108 from the first lateral side 107 of the patient support 102 as it extends upwardly from the base 121.

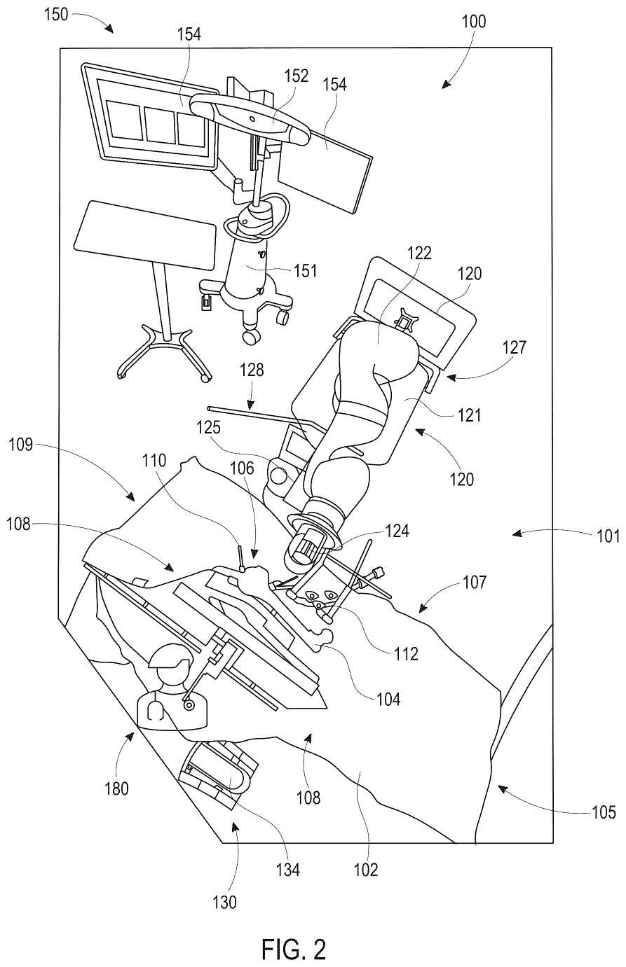

In some embodiments, as shown in FIGS. 2-6, the system 100 and/or patient support 102 may comprise a fixation assembly 108 that is configured to fix the position and/or orientation of the surgical site 106 of the patient 104 relative to the patient support 102. The fixation assembly 108 may comprise one or more clamp, pin, wire, band or other mechanism that securely couples the surgical site 106 (e.g., bones of a joint) relative to the patient support 102 during a cutting operation.

The active robotic cutting system 120 may be programmed to include a plurality of determined, prescribed or programmed cut paths of the cutting tool 129 to effectuate cut of the surgical site 106 along or according to the determined, prescribed or programmed cut paths. The active robotic cutting system 120 may include control software that dictates or instructs, inter alai, the articulated arm 122 of the active robotic cutting system 120 to adjust in particular ways (i.e., adjustment of the joints) to translate the cutting tool 129 in three-dimensional space along the determined, prescribed or programmed cut paths of the cutting tool 129 to cut the surgical site 106 along or according to the determined, prescribed or programmed cut paths. It is noted that the end effector 124 translates the cutting tool 129 along its cutting pathway while the active robotic cutting system 120 translates the cutting tool 129 (via the articulated arm 122, at least partially) along the determined, prescribed or programmed cut paths (which the user does not determine or effectuate, but rather are fully autonomously determined and/or performed by the active the system 100). It is also noted that the cut paths may be planned and tracked via the navigational system 150 and at least one fixed patient tracking array 110, 112 couples to the patient 104 at fixed positions and orientations relative to the surgical site 106, which are registered by the system 100 prior to a cutting operation).

The cut paths of the cutting tool 129 may thereby be predetermined, pre-prescribed or pre-programmed in/via the system 100 (e.g., the navigational system 150) such that the user 180 does not dictate the cut paths of the cutting tool 129 that the cutting tool 129 travels along to cut the surgical site 106. As explained further below, the user may be provided with control over parameters or metrics of the cutting tool 129 as it moves along the cut paths via a controller 130, but not the configuration or three-dimensional parameters of the cut paths themselves (i.e., the user does not determine or control the portions of the surgical site 106 (i.e., bone) that are removed/cut (i.e., the cut paths themselves). It is noted that the cut paths may thereby include entry positions where the cutting tool 129 first initiates cutting of the surgical site 106 and enters the surgical site 106, as well as three-dimensional spatial pathways extending from the entry positions through the surgical site 106, and represent removed or cut portions of the surgical site 106.

In some embodiments, the cut paths comprise spatial pathways to and through a surgical site 106 (e.g., bone). The cut paths may thereby be configured to resect or cut the surgical site 106 according to the requirement of a surgical procedure. In one example, the cut paths are configured to resect or cut a distal or proximal end portion of a bone surgical site 106 for the cooperation of an implant with the rested bone 106, such for a total knee arthroplasty (TKA) that involves resecting and replacing the articular surfaces of a tibia and/or femur (e.g., femoral condyles and tibial plateau).

As shown in FIGS. 1-6 and discussed above, the system 100 includes a navigational system 150 that is configured to image and track the positions and orientations of the cutting tool 129 and the surgical site 106, such as via the at least one fixed cutter tracking array 128 affixed to the active cutter system 120 and the at least one fixed patient tracking array 110, 112 affixed to the patient 104 near the surgical site 106. For example, the navigation system 150 may be configured to determine and track, via the at least one tracking camera 150 thereof, the three-dimensional position of the cutting edge based on a plurality of registration movements of the articulated arm 122 and the position and/or orientation of the cutting edge relative to the at least one fixed cutter tracking array 128 and/or camera 150. As another example, the navigation system 150 may be configured to determine and track the three-dimensional position of the cutting edge relative to the surgical site 106 via the at least one tracking camera 150 based on movement of the articulated arm 122 and the relative positions and orientations of the at least one fixed cutter tracking array 128 and/or the at least one fixed patient tracking array 110, 112. As also explained above, the navigation system 150 may be configured to determine and track, via the at least one tracking camera 150 and the at least one fixed patient tracking array 110, 112, the relative position and orientation of the surgical site (e.g., bones of a knee joint), and display representations or models thereof with digital planning features (e.g., implants and/or cut paths/zones) to digitally plan a surgical procedure. Further, during a cutting operation, the navigation system 150 may thereby also display at least one of a representation of the cutting tool and the surgical site based on the actual positions/interactions thereof.

As shown in FIGS. 1 and 2, the navigation system 150 is positioned adjacent to the first lateral side 107 of the patient support 102 and distally next to the active cutter system 120 (i.e., on the same lateral side of the patient support 102, patient 104 and surgical site 106). The navigation system 150 may be positioned further laterally away from the first lateral side 107 of the patient support 102 than the active cutter system 120. In some embodiments, as shown in FIG. 1, the navigation system 150 is positioned distally of the active cutter system 120, and/or distally of the surgical site 106. For example, the navigation system 150 may be positioned distally of the distal end 109 of the patient support 102, thereby providing an obstructed view for the camera system 152 of the patient 104 and/or surgical site 106. In this way, the at least one fixed cutter tracking array 128, the at least one fixed patient tracking array 110, 112 and/or the surgical site 106 can be positioned along a linear line of sight from the at least one camera 152. In some embodiments, the at least one fixed cutter tracking array 128 may be positioned distally of the base 121, and laterally from the at least one fixed patient tracking array 110, 112 and the surgical site 106 in a direction extending from the first to the second lateral sides 107, 108 of the patient support 102.

As noted above, the navigation system 150 may comprise a base 151, at least one tracking camera 152, at least one graphical display 154, and a computer tracking system configured to track the cutting tool 129, surgical site 106 (e.g., bones thereof), the at least one fixed patient tracking array 110, 112 and/or the at least one fixed cutter tracking array 128.

As with the base 121 of the active cutter system 120, the base 151 of the navigation system 150 may be configured as a movable cart base. For example, the base 151 (and/or base 131) may include lockable wheels that allow for selective movement or translation thereof. The base 151 (and/or base 131), and thereby the navigation system 150 (and/or the active cutter system 120) may thereby be configured such that it can be manually positioned and then locked in place via wheels/casters or the like.

The navigation system 150 may be effective in imaging the cutting tool 129 (or a registration version thereof), the at least one fixed patient tracking array 110, 112 and/or the at least one fixed cutter tracking array 128, so that the relative three-dimensional positions and orientations of the cutting tool 129 and the surgical site 106 can be determined/tracked to, ultimately, determine the necessary adjustments of the arm 122 to autonomously effectuate one or more cut paths via the cutting tool 129. In such embodiments, the active cutter system 120 may be void of any such tracking cameras, software or the like.

As shown in FIGS. 1-3, the system 100 may further include the user controller 130 in the surgical theater 101.

The user controller 130 may that comprises plurality of control inputs that a user can selectively actuate to control the movement of the cutting tool 129 to execute one of the determined, prescribed or programmed cut paths. In some embodiments, the plurality of control inputs comprise a plurality of manually engageable, activatable or actuatable electrical switches, sensors or the like configured to send an electronic signal or otherwise communicate with the active robotic cutting system 120 to provide the active robotic cutting system 120 with an action or instruction that alters or effects current cutting parameters of the active robotic cutting system 120. For example, the plurality of control inputs may comprise buttons, paddles, triggers, directional pads, thumb sticks, or the like, and related associated electrical circuitry and componentry.

As shown in FIGS. 1-3, the electronic user controller 130 positioned adjacent to the patient support 102 and thus the patient 102 and the surgical site 106 on the second lateral side 108. As also shown, the user controller 102 and the at least the base 121 of the active cutting system 120 may be substantially aligned in the proximal-distal direction. In some embodiments, the surgical site 106 is positioned distally of the user controller 130 and proximally of the navigation system 150, as shown in FIG. 1. The user controller 130 is configured to be operated by a user 180, such as a surgeon or doctor, as shown in FIG. 1,

In some such embodiments, the plurality of control inputs comprises at least one first control input configured such that, when selectively actuated by a user 180, active robotic cutting system 120 is directed to advance the cutting tool 129 along a current programmed cut path that active robotic cutting system 120 is executing. In some such embodiments, the plurality of control inputs comprises at least one second control input configured such that, when selectively actuated by a user 180, active robotic cutting system 120 is directed to retreat or reverse the cutting tool 129 along a current programmed cut path that active robotic cutting system 120 executed immediately previous thereto. In some embodiments, the plurality of control inputs comprises at least one third control input configured such that, when selectively actuated by a user 180, active robotic cutting system 120 is directed to translate the cutting tool 129 along a current programmed cut path that active robotic cutting system 120 is executing at an increased velocity or speed as compared to a current programmed (and effectuated) velocity thereof. In some embodiments, the plurality of control inputs comprises at least one fourth control input configured such that, when selectively actuated by a user 180, active robotic cutting system 120 is directed to translate the cutting tool 129 along a current programmed cut path that active robotic cutting system 120 is executing at a decreased velocity or speed as compared to a current programmed (and effectuated) velocity thereof. In some embodiments, the plurality of control inputs comprises at least one fifth control input configured such that, when selectively actuated by a user 180, active robotic cutting system 120 is directed to cease translating the cutting tool 129 along its cutting pathway. For example, the at least one fifth control input may shut of the end effector 124 or otherwise cause the end effector 124 to stop powering or translating the cutting tool 129 along its cutting pathway. In some embodiments, the plurality of control inputs comprises at least one sixth control input configured such that, when selectively actuated by a user 180, active robotic cutting system 120 is directed to register a current point or location of the cutting tool 129 along the cutting pathway that active robotic cutting system 120 is currently executing.

In some exemplary embodiments, the active robotic cutting system 120 and related methods may comprise or make use of an exemplary controller 130 that is configured as a foot operated controller such that the plurality of control inputs thereof are configured to be selectively actuated by a user's foot. In some such embodiments, as shown in FIGS. 1-3, the controller 130 may comprise a foot pedal 134 that comprises an engagement side or surface that is configured to be selectively engaged by the underside of a user's foot, and a housing that movably supports the foot pedal. The housing may comprise a base portion that is configured to support the controller 130 and engage a ground surface, and one or more side or peripheral portions that are positioned adjacent (e.g., medially, laterally, proximally and/or distal) to the foot pedal 134.

The foot pedal 134 may be pivotably or articulably coupled to the housing. The foot pedal 134 may be configured such that a user 180 can articulate the foot pedal 134 between a first forefoot position with a forefoot portion of the foot pedal 134 being depressed or positioned in a depressed or lowered location as compared to a hindfoot portion of the foot pedal 134 (not shown), and a second hindfoot position with the hindfoot portion of the foot pedal 134 being depressed or positioned in a depressed or lowered location as compared to the forefoot portion 58 of the foot pedal 134. The forefoot portion of the foot pedal 134 may be configured to be positioned beneath and engage a forefoot portion of a user's foot, and the hindfoot portion of the foot pedal 134 may be configured to be positioned beneath and engage a hindfoot portion of the user's foot.

In some embodiments, the first forefoot position of the foot pedal 134 actuates a first control input that is configured to direct or control the active robotic cutting system 120 to advance the cutting tool 129 along a current programmed cut path that the active robotic cutting system 120 is executing when the first control input is selectively actuated by a user 180 (via the first forefoot position of the foot pedal 134). In some embodiments, the second hindfoot position of the foot pedal 134 actuates a second control input that is configured to direct or control the active robotic cutting system 120 to retreat or reverse the translation of the cutting tool 129 along a current programmed cut path that the active robotic cutting system 120 executed immediately previous thereto when the second control input is selectively actuated by the user 180 (via the second hindfoot position of the foot pedal 134).

In some embodiments, the foot pedal 134 is configured such that when positioned in a neutral position between the first forefoot position and the second hindfoot position, the controller 130 directs the active robotic cutting system 120 to stop or pause the cutting tool 129 in its current position along a current programmed cut path. In some embodiments, the foot pedal 134 may be configured to be selectively rotated along a medial-lateral direction 66 by a user's foot. The controller 130 may be configure such that medial-lateral rotation of the foot pedal 134 activates at least one sixth control input that, when selectively actuated by a user 180 via rotation of the foot pedal 134, directs the active robotic cutting system 120 to register a current point of the cutting tool 129 along the cutting pathway that the active robotic cutting system 120 is executing.

The housing of the foot controller 130 may comprise a plurality of the plurality of control inputs that are configured to be selectively engaged by the user's foot. For example, in some embodiments, a first portion of the housing may comprises a third control input that, when selectively actuated by a user's foot, is configured to direct the active robotic cutting system 120 to translate the cutting tool 129 along a current programmed cut path that the active robotic cutting system 120 is executing at an increased speed or velocity as compared to a current programmed velocity. Similarly, in some embodiments, a second portion of the housing may comprises a fourth control input that, when selectively actuated by a user's foot, is configured to direct the active robotic cutting system 120 to translate the cutting tool 129 along a current programmed cut path that the active robotic cutting system 120 is executing at a decreased speed or velocity as compared to a current programmed velocity. In some embodiments, the first and second portions of the housing may be on opposing sides of the foot pedal 134, such as positioned on medial and lateral sides thereof.

In some embodiments, the housing may comprise a fourth control input that, when selectively actuated by a user's foot, is configured to direct the active robotic cutting system 120 to stop or pause the end effector 124 from translating the cutting tool 129 along its cutting pathway. In some such embodiments, the fourth control input may also be configured such that when selectively actuated by a user's foot, the active robotic cutting system 120 is directed to stop or pause the cutting tool 129 in its current position along a current programmed cut path.

In some exemplary embodiments, the active robotic cutting system 120 and related methods may comprise or make use of an exemplary controller that is configured as a hand operated controller (not shown). The controller may be configured as a handheld controller such that the plurality of control inputs are configured to be selectively actuated by one or more fingers of a hand of the user 180 similar to that of the foot controller 130.

As with the at least one fixed cutter tracking array 128 and the base 121 (and/or cutting blade 129) of the active cutting system 120, the at least one fixed patient tracking array 110, 112 is utilized by the system 100, and in particular the navigational system 150, to determine and track the position and orientation of the surgical site 106 in three-dimensional space. The at least one fixed patient tracking array 110, 112 thereby comprises a plurality of identifiably-shaped portions, and is configured to be affixed to the surgical site 106 at predefined registered relative positions and orientations with respect to the surgical site 106. Once the at least one fixed patient tracking array 110, 112 and the surgical site 106 are registered via the system 100, the position and orientation of the at least one fixed patient tracking array 110, 112 allows the navigational system 150 to determine and track, and graphically represent on the screen 154, the position and orientation of the surgical site 106 (bones of a joint). The at least one fixed patient tracking array 110, 112 may also be utilized by the navigational system 150 to determine the relative position and orientation of the base 121 of the active cutting system 120 relative to the surgical site 106.

In some embodiments, the at least one fixed patient tracking array comprises a first fixed patient tracking array 110 configured to affix to a first anatomical structure related to the surgical site 106, and a second fixed patient tracking array 112 configured to affix to a second anatomical structure related to the surgical site 106 that differs from the first anatomical structure. In some such embodiments, the first and second anatomical structures may be separate bones, such as a proximal tibia and distal femur of a knee joint.

As shown in FIGS. 1-3, the at least one fixed patient tracking array 110, 112 may face laterally toward the first lateral side 107 in a lateral direction extending from the second side 108 to the first side 107 of the patient support 102, and potentially distally. For example, at least the identifiably-shaped and/or colored portions toward the first lateral side 107 and/or distally, and the active cutting system 120 may be configured to perform cuts at least generally in an opposing lateral direction extending from the first side 107 to the second side 108 of the patient support 102.

It is to be understood that the above description is intended to be illustrative, and not restrictive. For example, the above-described exemplary embodiments, and/or aspects thereof, may be used in combination with each other. In addition, many modifications may be made to adapt a particular configuration according to the teachings of the various examples without departing from their scope.

Processes described herein may be performed singly or collectively by one or more computer systems, such as one or more systems that are, or are in communication with, the robotic system, such as the articulating joints, end effector, controller, camera system, tracking system, and/or AR system thereof, as examples. FIG. 7 depicts one example of such a computer system and associated devices to incorporate and/or use aspects described herein. A computer system may also be referred to herein as a data processing device/system, computing device/system/node, or simply a computer. The computer system may be based on one or more of various system architectures and/or instruction set architectures, such as those offered by Intel Corporation (Santa Clara, California, USA) or ARM Holdings plc (Cambridge, England, United Kingdom), as examples.

FIG. 7 shows a computer system 200 in communication with external device(s) 212. Computer system 200 includes one or more processor(s) 202, for instance central processing unit(s) (CPUs). A processor can include functional components used in the execution of instructions, such as functional components to fetch program instructions from locations such as cache or main memory, decode program instructions, and execute program instructions, access memory for instruction execution, and write results of the executed instructions. A processor 202 can also include register(s) to be used by one or more of the functional components. Computer system 200 also includes memory 204, input/output (I/O) devices 208, and I/O interfaces 210, which may be coupled to processor(s) 202 and each other via one or more buses and/or other connections. Bus connections represent one or more of any of several types of bus structures, including a memory bus or memory controller, a peripheral bus, an accelerated graphics port, and a processor or local bus using any of a variety of bus architectures. By way of example, and not limitation, such architectures include the Industry Standard Architecture (ISA), the Micro Channel Architecture (MCA), the Enhanced ISA (EISA), the Video Electronics Standards Association (VESA) local bus, and the Peripheral Component Interconnect (PCI).

The memory 204 can be or include main or system memory (e.g., Random Access Memory) used in the execution of program instructions, storage device(s) such as hard drive(s), flash media, or optical media as examples, and/or cache memory, as examples. Memory 204 can include, for instance, a cache, such as a shared cache, which may be coupled to local caches (examples include L1 cache, L2 cache, etc.) of processor(s) 202. Additionally, the memory 204 may be or include at least one computer program product having a set (e.g., at least one) of program modules, instructions, code or the like that is/are configured to carry out functions of embodiments described herein when executed by one or more processors.

The memory 204 can store an operating system 205 and other computer programs 206, such as one or more computer programs/applications that execute to perform aspects described herein. Specifically, programs/applications can include computer readable program instructions that may be configured to carry out functions of embodiments of aspects described herein.

Examples of I/O devices 208 include but are not limited to microphones, speakers, Global Positioning System (GPS) devices, RGB and/or IR cameras, lights, accelerometers, gyroscopes, magnetometers, sensor devices configured to sense light, proximity, heart rate, body and/or ambient temperature, blood pressure, and/or skin resistance, registration probes and activity monitors. An I/O device may be incorporated into the computer system as shown, though in some embodiments an I/O device may be regarded as an external device (212) coupled to the computer system through one or more I/O interfaces 210.

The computer system 200 may communicate with one or more external devices 212 via one or more I/O interfaces 210. Example external devices include a keyboard, a pointing device, a display, and/or any other devices that enable a user to interact with the computer system 200. Other example external devices include any device that enables the computer system 200 to communicate with one or more other computing systems or peripheral devices such as a printer. A network interface/adapter is an example I/O interface that enables the computer system 200 to communicate with one or more networks, such as a local area network (LAN), a general wide area network (WAN), and/or a public network (e.g., the Internet), providing communication with other computing devices or systems, storage devices, or the like. Ethernet-based (such as Wi-Fi) interfaces and Bluetooth® adapters are just examples of the currently available types of network adapters used in computer systems (BLUETOOTH is a registered trademark of Bluetooth SIG, Inc., Kirkland, Washington, U.S.A.).

The communication between I/O interfaces 210 and the external devices 212 can occur across wired and/or wireless communications link(s) 211, such as Ethernet-based wired or wireless connections. Example wireless connections include cellular, Wi-Fi, Bluetooth®, proximity-based, near-field, or other types of wireless connections. More generally, the communications link(s) 211 may be any appropriate wireless and/or wired communication link(s) for communicating data.

The particular external device(s) 212 may include one or more data storage devices, which may store one or more programs, one or more computer readable program instructions, and/or data, etc. The computer system 200 may include and/or be coupled to and in communication with (e.g., as an external device of the computer system) removable/non-removable, volatile/non-volatile computer system storage media. For example, it may include and/or be coupled to a non-removable, non-volatile magnetic media (typically called a “hard drive”), a magnetic disk drive for reading from and writing to a removable, non-volatile magnetic disk (e.g., a “floppy disk”), and/or an optical disk drive for reading from or writing to a removable, non-volatile optical disk, such as a CD-ROM, DVD-ROM or other optical media.

The computer system 200 may be operational with numerous other general purpose or special purpose computing system environments or configurations. Computer system 200 may take any of various forms, well-known examples of which include, but are not limited to, personal computer (PC) system(s), server computer system(s), such as messaging server(s), thin client(s), thick client(s), workstation(s), laptop(s), handheld device(s), mobile device(s)/computer(s) such as smartphone(s), tablet(s), and wearable device(s), multiprocessor system(s), microprocessor-based system(s), telephony device(s), network appliance(s) (such as edge appliance(s)), virtualization device(s), storage controller(s), set top box(es), programmable consumer electronic(s), network PC(s), minicomputer system(s), mainframe computer system(s), and distributed cloud computing environment(s) that include any of the above systems or devices, and the like.

Various input devices may be provided, such as a camera, which can be used to capture images or video. The camera can be used by the device to obtain image(s)/video of a view of the material to be cut and/or the cutting tool, for instance, capturing images/videos of a scene.

One or more microphones, proximity sensors, light sensors, accelerometers, speakers, GPS devices, and/or other input devices (not labeled) may be additionally provided. Electronic components, such as electronic circuitry, including processor(s), memory, and/or communications devices, such as cellular, short-range wireless (e.g., Bluetooth), or Wi-Fi circuitry for connection to remote devices may be included. A power source, such as a battery to power components of the system may also be incorporated. Physical port(s) (not pictured) used to connect device the computer to a power source (to recharge a battery) and/or any other external device, such as the controller. Such physical ports can be of any standardized or proprietary type, such as Universal Serial Bus (USB).

Aspects of the present invention may be a system, a method, and/or a computer program product, any of which may be configured to perform or facilitate aspects described herein.

In some embodiments, aspects of the present invention may take the form of a computer program product, which may be embodied as computer readable medium(s). A computer readable medium may be a tangible storage device/medium having computer readable program code/instructions stored thereon. Example computer readable medium(s) include, but are not limited to, electronic, magnetic, optical, or semiconductor storage devices or systems, or any combination of the foregoing. Example embodiments of a computer readable medium include a hard drive or other mass-storage device, an electrical connection having wires, random access memory (RAM), read-only memory (ROM), erasable-programmable read-only memory such as EPROM or flash memory, an optical fiber, a portable computer disk/diskette, such as a compact disc read-only memory (CD-ROM) or Digital Versatile Disc (DVD), an optical storage device, a magnetic storage device, or any combination of the foregoing. The computer readable medium may be readable by a processor, processing unit, or the like, to obtain data (e.g., instructions) from the medium for execution. In a particular example, a computer program product is or includes one or more computer readable media that includes/stores computer readable program code to provide and facilitate one or more aspects described herein.

As noted, program instruction contained or stored in/on a computer readable medium can be obtained and executed by any of various suitable components such as a processor of a computer system to cause the computer system to behave and function in a particular manner. Such program instructions for carrying out operations to perform, achieve, or facilitate aspects described herein may be written in, or compiled from code written in, any desired programming language. In some embodiments, such programming language includes object-oriented and/or procedural programming languages such as C, C++, C#, Java, etc.

The program code can include one or more program instructions obtained for execution by one or more processors. The computer program instructions may be provided to one or more processors of, e.g., one or more computer systems, to produce a machine, such that the program instructions, when executed by the one or more processors, perform, achieve, or facilitate aspects of the present invention, such as actions or functions described in flowcharts and/or block diagrams described herein. Thus, each block, or combinations of blocks, of the flowchart illustrations and/or block diagrams depicted and described herein can be implemented, in some embodiments, by computer program instructions.

Many other examples will be apparent to those of skill in the art upon reviewing the above description. The scope of the various examples should, therefore, be determined with reference to the appended claims, along with the full scope of equivalents to which such claims are entitled.

While dimensions and types of materials may be described herein, they are intended to define parameters of some of the various examples, and they are by no means limiting to all examples and are merely exemplary.

In the appended claims, the terms “including” and “in which” are used as the plain-English equivalents of the respective terms “comprising” and “wherein.” Moreover, in the following claims, the terms “first,” “second,” and “third,” etc. are used merely as referee labels, and are not intended to impose numerical, structural or other requirements on their objects. Forms of term “based on” herein encompass relationships where an element is partially based on as well as relationships where an element is entirely based on. Forms of the term “defined” encompass relationships where an element is partially defined as well as relationships where an element is entirely defined. Further, the limitations of the following claims are not written in means-plus-function format and are not intended to be interpreted based on 35 U.S.C. § 112, sixth paragraph, unless and until such claim limitations expressly use the phrase “means for” followed by a statement of function cavity of further structure. It is to be understood that not necessarily all such objects or advantages described above may be achieved in accordance with any particular example. Thus, for example, those skilled in the art will recognize that the devices, systems and methods described herein may be embodied or carried out in a manner that achieves or optimizes one advantage or group of advantages as taught herein without necessarily achieving other objects or advantages as may be taught or suggested herein.

While the disclosure has been described in detail in connection with only a limited number of examples, it should be readily understood that the disclosure is not limited to such disclosed examples. Rather, this disclosure can be modified to incorporate any number of variations, alterations, substitutions or equivalent arrangements not heretofore described, but which are commensurate with the spirit and scope of the disclosure. Additionally, while various examples have been described, it is to be understood that aspects of the disclosure may include only one example or some of the described examples. Also, while some disclosure are described as having a certain number of elements, it will be understood that the examples can be practiced with less than or greater than the certain number of elements.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

What is claimed is:

1. A surgical robotic system, comprising:
a patient support configured to physically support a surgical site of a patient at a particular location;
an active cutting system positioned adjacent to the patient support on a first lateral side of the patient support comprising a base, at least one fixed cutter tracking array, a powered articulated arm coupled to the base, an end effector coupled to an end arm segment of the articulated arm, and a cutting tool coupled by the end effector defining a cutting edge;
an electronic user controller control comprising a plurality of manually-operable control inputs that are configured to control operational parameters of the cutting tool, the electronic controller positioned adjacent to the patient support on a second lateral side of the patient support that opposes the first lateral side of the patient support;
at least one fixed patient tracking array configured to couple to the patient at a fixed positional relationship to a surgical site of the patient; and
a navigation system positioned on the first lateral side of the patient support, comprising a base, at least one tracking camera, at least one graphical display, and a computer tracking system configured to determine the three-dimensional position of the cutting edge via the at least one tracking camera based on at least one of movement of the articulated arm, the at least one fixed cutter tracking array, and the relative at least one fixed patient tracking array.

2. The system according to claim 1, wherein the system is configured such that the at least one fixed cutter tracking array is positioned distally of the base and the articulated arm of the active cutting system.

3. The system according to claim 2, wherein the system is configured such that the at least one fixed cutter tracking array is positioned within a line of sight between the at least one camera and the surgical site as positioned by the patient support.

5. The system according to claim 3, wherein the at least one fixed cutter tracking array comprises a plurality of identifiably-shaped portions at predefined relative positions and orientations with respect to each other.

6. The system according to claim 1, wherein the system is configured such that the at least one fixed cutter tracking array is positioned along a linear line of sight between the at least one camera and the surgical site as positioned by the patient support.

7. The system according to claim 1, wherein the at least one fixed patient tracking array comprises a plurality of identifiably-shaped portions at predefined registered relative positions and orientations with respect to the surgical site when affixed thereto.

8. The system according to claim 7, wherein the at least one fixed patient tracking array comprises a first fixed patient tracking array configured to affix to a first anatomical structure related to the surgical site, and a second fixed patient tracking array configured to affix to a second anatomical structure related to the surgical site that differs from the first anatomical structure.

9. The system according to claim 8, wherein the first anatomical structure is a distal femur, and the second anatomical structure surgical site is a proximal tibia.

10. The system according to claim 1, wherein the surgical site is a portion of an articular surface of a tibia and/or an articular surface femur of a knee joint.

11. The system according to claim 1, wherein the at least one fixed patient tracking array faces laterally toward the first lateral side and distally.

12. The system according to claim 1, wherein the system is configured such that the surgical site is positioned distally of the user controller and proximally of the navigation system.

13. The system according to claim 12, wherein the system is configured such that the surgical site is positioned proximate to the second lateral side of the patient support and distal to the first lateral side of the patient support.

14. The system according of claim 13, wherein the system is configured such that the articulated arm extends over the patient and the patient support to proximate to the surgical site from the first lateral side of the patient support laterally to the surgical site.

15. The system according to claim 1, wherein the system is configured such that the surgical site is positioned proximate to the second lateral side of the patient support and distal to the first lateral side of the patient support.

16. The system according of claim 1, wherein the system is configured such that the articulated arm extends over the patient and the patient support to proximate to the surgical site from the first lateral side of the patient support laterally to the surgical site.

17. The system according to claim 1, wherein the base of the active cutting system is a movable cart.

18. The system according to claim 1, wherein a base arm segment of the articulated arm is coupled to the base of the active cutting system at an angle that extends laterally toward the second lateral side of the patient support as it extends upwardly from the base.

19. The system according to claim 1, wherein the system is configured such that the navigation system is positioned on the first lateral side of the patient support distally past a distal end of the patient support.

20. The system according to claim 1, wherein the system is configured such that the user controller and the base of the active cutting system are substantially aligned in the proximal-distal direction.

21. The system according to claim 1, wherein the system is configured such that:
the at least one fixed cutter tracking array is positioned distally of the base and the articulated arm of the active cutting system;
the at least one fixed cutter tracking array is positioned within a line of sight between the at least one camera and the surgical site as positioned by the patient support;
the surgical site is positioned distally of the user controller and proximally of the navigation system;
the surgical site is positioned proximate to the second lateral side of the patient support and distal to the first lateral side of the patient support; and
the articulated arm extends over the patient and the patient support to proximate to the surgical site from the first lateral side of the patient support laterally to the surgical site.

22. The system according to claim 21, wherein the system is further configured such that the navigation system is positioned on the first lateral side of the patient support distally past a distal end of the patient support.

23. The system according to claim 22, wherein the system is further configured such that the user controller and the base of the active cutting system are substantially aligned in the proximal-distal direction.

24. The system according to claim 1, wherein the base of the navigation system is a movable cart.

25. The system according to claim 1, wherein the system is configured such that the plurality of control inputs control the operation of the cutting tool while executing at least one programmed cut path but do not alter the configuration of the at least one programmed cut path.

26. The system according to claim 1, wherein the robotic system is configured to autonomously adjust the relative orientation of the arm segments of the articulated arm to autonomously spatially translate the cutting tool and cut material at the surgical site along at least one programmed cut path.

27. The system according to claim 26, wherein the system is configured such that the plurality of control inputs control the operation of the cutting tool while executing the at least one programmed cut path but do not alter the configuration of the at least one programmed cut path.

28. The system according to claim 1, wherein the navigation system is configured to display at least one of a representation of the cutting tool and the surgical site based on the actual positions thereof.

29. The system according to claim 1, wherein the active cutting system further comprises at least one graphical display positioned at a back lateral side of the active cutting system that faces laterally away from the patient support, and wherein the active cutting system is configured to display a representation of at least one of at least one preprogrammed cut path and the position of the cutting tool relative to the at least one preprogrammed cut path and/or the surgical sight.

30. The system according to claim 1, wherein the cutting edge of the cutting tool is configured to cut while being translated along a cutting pathway, and wherein the end effector comprises a powered drive portion that translates the cutting tool along the cutting pathway.

31. The system according to claim 1, wherein the navigation system is configured to determine and track the three-dimensional position of the cutting edge in consideration of a plurality of registration movements of the articulated arm and the position and/or orientation of the cutting edge relative to that of the at least one tracking camera.

32. The system according to claim 1, wherein the navigation system is configured to determine and track the three-dimensional position of the cutting edge relative to the surgical site via the at least one tracking camera based on movement of the articulated arm and the relative positions and orientations of the at least one fixed cutter tracking array and the at least one fixed patient tracking array.

33. The system according to claim 1, wherein the plurality of control inputs comprises at least one first control input that, when selectively actuated by a user, is configured to direct the robotic system to advance or retract the cutting tool along a current programmed cut path that the robotic system is executing.

34. The system according to claim 33, wherein the plurality of control inputs comprises at least one second control input that, when selectively actuated by a user, is configured to direct the robotic system to translate the cutting tool along a current programmed cut path that the robotic system is executing at an increased or decreased velocity as compared to a current programmed velocity.

35. The system according to claim 34, wherein the plurality of control inputs comprises at least one control input that, when selectively actuated by a user, is configured to stop the end effector from translating the cutting tool along the cutting pathway.

36. The system according to claim 35, wherein the controller is configured as a foot controller such that the plurality of control inputs are configured to be selectively actuated by a user's foot.

37. The system according to claim 1, wherein the controller is configured as a foot controller such that the plurality of control inputs are configured to be selectively actuated by a user's foot.

38. The system according to claim 37, wherein the foot controller comprises a foot pedal that is configured to be selectively engaged by the underside of a user's foot and a housing positioned adjacent to at least one side of the foot pedal, and wherein the foot pedal is configured such that the user can articulate the foot pedal between a first forefoot position with a forefoot portion of the foot pedal being depressed, and a second hindfoot position with a hindfoot portion of the foot pedal being depressed.

39. The system according to claim 38, wherein the first forefoot position of the foot pedal actuates a first control input that is configured to direct the robotic system to advance the cutting tool along a current programmed cut path that the robotic system is executing when selectively actuated by the user.

40. The system according to claim 38, wherein the second hindfoot position of the foot pedal actuates a second control input second control input that is configured to direct the robotic system to retreat the cutting tool along a current programmed cut path that the robotic system executed immediately previous thereto when selectively actuated by the user.

41. The system according to claim 38, wherein the housing comprises a plurality of the plurality of control inputs that are configured to be selectively engaged by the user's foot.

42. The system according to claim 1, wherein the cutting tool is a sagittal cutting blade with cutting teeth positioned at a longitudinal end thereof, the sagittal cutting blade being configured to cut when translated longitudinally and oscillated along a cutting pathway that lies in a plane in which the blade is aligned about an axis of oscillation.

43. The system according to claim 42, wherein the cutting pathway is at least one programmed cut path that extends through a first bone of the patient such that the cutting tool cuts the first bone when executing the least one programmed cut path.

44. The system according to claim 43, wherein the robotic system is configured to autonomously adjust the relative orientation of the arm segments of the articulated arm to autonomously spatially translate the cutting tool along the least one programmed cut path.

45. The system according to claim 44, wherein the system is configured such that the plurality of control inputs control the operation of the cutting tool while executing the at least one programmed cut path but do not alter the configuration of the at least one programmed cut path.

46. The system according to claim 1, wherein the patient support comprises a fixation assembly that is configured to fix the position and/or orientation of the surgical site of the patient relative to the patient support.

47. A method of cutting material at a surgical site, comprising:
utilizing the system according to claim 1 to autonomously translate the cutting tool along a least one programmed cut path cut material at the surgical site.
